(19) United States
(12) Patent Application Publication
(10) Pub. No.: US 2005/0096259 A1
(43) Pub. Date: May 5, 2005

(54) Neutrophil activation by immune response modifier compounds

(75) Inventors: Mark A. Tomai, Woodbury, MN (US); John P. Vasilakos, Woodbury, MN (US); Paul D. Wightman, Woodbury, MN (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 10/978,850

(22) Filed: Nov. 1, 2004

Related U.S. Application Data
(60) Provisional application No. 60/516,116, filed on Oct. 31, 2003.
(60) Provisional application No. 60/517,805, filed on Nov. 6, 2003.

(51) Int. Cl. A61K 38/00

(52) U.S. Cl. 514/2

(57) ABSTRACT

The invention provides a method of activating neutrophils. Generally, the method includes contacting neutrophils with a neutrophil-activating IRM compound and/or a TLR8-selective agonist in an amount effective to activate the neutrophils. In some embodiments, the method may be used to treat a condition treatable by activating neutrophils. In another aspect, the invention provides pharmaceutical compositions that generally include a neutrophil-activating IRM compound and/or a TLR8-selective agonist, or a pharmaceutically acceptable form thereof, in an amount effective to activate neutrophils.

### CROSS-REFERENCE TO RELATED APPLICATION

This application claims priority to U.S. Provisional Patent Application Ser. No. 60/516,116, filed Oct. 31, 2003, and U.S. Provisional Patent Application Ser. No. 60/517,805, filed Nov. 6, 2003.

### BACKGROUND

Neutrophils are the most abundant immune cells in human blood. However, when infection occurs, neutrophils migrate from the bloodstream to the site of infection and contribute to the primary immunological defense. Neutrophils produce antimicrobial products and proinflammatory cytokines that can promote containment of the infection, which can provide the acquired immune system with enough time to clear the infection and generate immunological memory. Neutrophils, as well as other professional phagocytes including, for example, macrophages, clear many bacterial infections.

Toll-like receptors (TLRs) are transmembrane receptors involved in innate immune recognition of pathogens. Human neutrophils express most of the human TLRs: TLRs 1, 2, 4, 5, 6, 7, 8, 9, and 10. Signaling through certain TLRs can activate neutrophils, which can trigger neutrophils to perform their various functions in generating an immune response to an infection. Thus, agonists of certain TLRs have been identified as stimulators of human neutrophil function.

In view of the therapeutic potential for activating neutrophils in a course of treatment for certain types of conditions (e.g., extracellular infections and neoplastic conditions), there is a substantial ongoing need to identify additional substances that can activate neutrophils.

### SUMMARY

It has been found that certain IRM compounds can be used to activate neutrophils. Suitable IRM compounds include, for example, TLR8-slective agonists and/or substituted imidazoquinoline amines. Accordingly, the present invention provides a method of activating neutrophils in which the method generally includes contacting neutrophils with a TLR8-selective agonist and/or a substituted imidazoquinoline amine in an amount sufficient to activate the neutrophils. In some embodiments, the neutrophils may be activated in vitro. In alternative embodiments, the neutrophils may be activated in vivo.

In another aspect, the present invention also provides a method of treating a condition in a subject. Generally, the method includes administering a TLR8-selective agonist and/or substituted imidazoquinoline amine to neutrophils of the subject in an amount effective to activate the subject's neutrophils sufficiently to treat the condition. In some embodiments, the subject's neutrophils may be activated in vitro, while in alternative embodiments the subject's neutrophils may be activated in vivo. When the subject's neutrophils are activated in vitro, the activated neutrophils may be re-introduced into the subject.

In another aspect, the present invention provides pharmaceutical compositions that generally include a TLR8-selective agonist and/or a substituted imidazoquinoline amine, or a pharmaceutically acceptable form thereof.

Various other features and advantages of the present invention should become readily apparent with reference to the following detailed description, examples, claims and appended drawings. In several places throughout the specification, guidance is provided through lists of examples. In each instance, the recited list serves only as a representative group and should not be interpreted as an exclusive list.

### BRIEF DESCRIPTION OF THE DRAWING

FIG. 1 shows IL-8 production by human neutrophils upon stimulation with TLR agonists.

### DETAILED DESCRIPTION OF ILLUSTRATIVE EMBODIMENTS OF THE INVENTION

Neutrophils are important components of innate immunity. Activated neutrophils can kill microbes that have entered a host. Left unchecked, the microbes can establish an infection that, depending upon the microbe, the host, and many other factors, can cause illness or, in severe cases, death. Enhancing the activation of neutrophils can enhance a host's early innate immune defenses against infection.

The present invention provides a method of activating neutrophils with, generally, a neutrophil-activating IRM compound. Thus, is some embodiments, the invention includes activating neutrophils using a neutrophil-activating IRM compound and a method of treating a condition in a subject using a neutrophil-activating IRM compound. In another aspect, the invention provides pharmaceutical compositions that include a neutrophil-activating IRM compound. This is the first demonstration of direct neutrophil activation using a neutrophil-activating IRM compound.

In some embodiments, the neutrophil-activating IRM compound can be a TLR8-selective agonist. Thus, is some embodiments, the invention includes activating neutrophils using a TLR8-selective agonist and a method of treating a condition in a subject using a TLR8-selective agonist. In another aspect, the invention provides pharmaceutical compositions that include a TLR8-selective agonist. This is the first demonstration of direct neutrophil activation using a TLR8-selective agonist. Thus, neutrophils may be directly activated using a compound that does not also act as a TLR7 agonist, thereby avoiding possibly undesirable effects that can result from activating TLR7-mediated biological activity.

As used herein, the term “TLR8-selective agonist” refers to any compound that, in an appropriate assay, can be demonstrated to act as an agonist of TLR8, but does not act as an agonist of TLR7. A TLR8-selective agonist may, therefore, act as an agonist for TLR8 and one or more of TLR1, TLR2, TLR3, TLR4, TLR5, TLR6, TLR9, or TLR10. Accordingly, while a TLR8-selective agonist may be a compound that acts as an agonist for TLR8 and for no other TLR, it may alternatively be a compound that acts as an agonist of TLR8 and, for example, TLR6.

As used with respect to the present invention, an agonist of a TLR refers to a compound that, when combined with the TLR, can produce a TLR-mediated cellular response. A compound may be considered an agonist of a TLR regardless of whether the compound can produce a TLR-mediated cellular response by (a) directly binding to the TLR, or (b) combining with the TLR indirectly by, for example, forming a complex with another molecule that directly binds to the TLR, or otherwise resulting in the modification of another compound so that the other compound can directly bind to the TLR.

The TLR agonism for a particular compound may be assessed in any suitable manner. For example, assays for detecting TLR agonism of test compounds are described, for example, in U.S. Patent Publication No. U.S. 2004/0132079, and recombinant cell lines suitable for use in such assays are described, for example, in International Patent Publication No. WO 04/053057. The assay used to assess the agonism of a compound with respect to one TLR may be the same as, or a different than, the assay used to assess the agonism of the compound with respect to another TLR.

Regardless of the particular assay employed, a compound can be identified as an agonist of TLR8 if performing the assay with a compound results in at least a threshold increase of some TLR8-mediated biological activity. Similarly, a compound may be identified as not acting as a TLR7 agonist (i.e., a TLR7 non-agonist) if, when used to perform an assay designed to detect TLR7-mediated biological activity, the compound fails to elicit a threshold increase in TLR7-mediated biological activity. Unless otherwise indicated, an increase in biological activity refers to an increase in the same biological activity over that observed in an appropriate control. An assay may or may not be performed in conjunction with the appropriate control. With experience, one skilled in the art may develop sufficient familiarity with a particular assay (e.g., the range of values observed in an appropriate control under specific assay conditions) that performing a control may not always be necessary to determine the TLR agonism of a compound in a particular assay.

The precise threshold increase of TLR-mediated biological activity for determining whether a particular compound is or is not an agonist of a particular TLR in a given assay may vary according to factors known in the art including but not limited to the biological activity observed as the endpoint of the assay, the method used to measure or detect the endpoint of the assay, the signal-to-noise ratio of the assay, the precision of the assay, and whether the same assay is being used to determine the agonism of a compound for both TLR7 and TLR8. Accordingly, it is not practical to set forth generally the threshold increase of TLR-mediated biological activity required to identify a compound as being an agonist or a non-agonist of a particular TLR for all possible assays. Those of ordinary skill in the art, however, can readily determine the appropriate threshold with due consideration of such factors.

Assays employing HEK293 cells transfected with an expressible TLR structural gene may use a threshold of, for example, at least a three-fold increase in a TLR-mediated biological activity (e.g., NFKB activation) when the compound is provided at a concentration of, for example, from about 1 μM to about 30 μM for identifying a compound as an agonist of the TLR transfected into the cell. However, different thresholds and/or different concentration ranges may be suitable in certain circumstances. Also, different thresholds may be appropriate for different assays.

In one aspect, the present invention provides a method of activating neutrophils. Generally, the method includes contacting neutrophils with an IRM compound, whether a TLR8-selective agonist in an amount effective to activate the neutrophils. Neutrophils may be activated either in vivo or in vitro.

When the neutrophils are activated in vitro, neutrophils may be collected from a source, contacted with the IRM compound in vitro, thereby activating at least a portion of the neutrophils in the sample, and then introduced into a subject. In some embodiments, the source of the neutrophils and the subject may be the same individual. In other embodiments, the source of the neutrophils and the subject may be different individuals.

A sample collected from the source may include cells other than neutrophils. Accordingly, the sample may be enriched for neutrophils or otherwise processed before the neutrophils are activated. Alternatively, the IRM compound may be administered to an unprocessed sample. Activated neutrophils may be washed or otherwise processed before being introduced into the subject. In alternative embodiments, unprocessed, activated neutrophils may be introduced into the subject. Depending upon the composition of the original sample, and the degree to which the sample is processed between collection from the source and introduction into the subject, the cells introduced into the subject may include cells other than neutrophils.

An amount of an IRM compound effective for activating neutrophils is an amount sufficient to increase at least one biological activity characteristic of activated neutrophils. Such biological activities include, for example, phagocytosis; production of cytokines and/or chemokines such as, for example, MIP-1α, MIP-1β, MIP-3α, GRO-α, IL-1β, or IL-8; chemotactic response to IL-8; shedding of L-selectin; generation of superoxide or other oxygen radicals associated with the respiratory burst; and decreased expression of certain chemokine receptors (e.g., CXCR1 or CXCR2).

The IRM compound may activate any suitable portion of neutrophils in the sample. In some embodiments, the IRM compound can activate from about 1% to about 100% of the neutrophils in the sample, although the methods of the present invention may be performed while activating a percentage of the neutrophils in the sample outside this range. In some embodiments, the IRM compound may activate at least about 80% of the neutrophils in the sample. In other embodiments, the IRM compound may activate at least about 50% of the neutrophils in the sample. In certain embodiments, the IRM compound may activate at least about 1% of the neutrophils in the sample, for example, at least about 10% of the neutrophils or from about 1% to about 5% of the neutrophils in the sample. In certain embodiments, a relatively low percentage (e.g., from about 1% to about 5%) of activated neutrophils may be obtained, but still provide practical utility because of the nature of a particular biological activity characteristic of activated neutrophils. For example, cell signaling such as through cytokine secretion can amplify biological activity downstream of the signal. Thus, a relatively small percentage of activated neutrophils may produce and secrete sufficient cytokine to induce a practical, useful level of biological activity in immune cells that are induced by (i.e., downstream of) the cytokine signal produced and secreted by the activated neutrophils.

When the neutrophils are activated in vivo, the IRM compound may be administered as a component of a pharmaceutical composition. Pharmaceutical compositions that include an IRM compound and methods of administering such pharmaceutical compositions are described in detail below.

Activated neutrophils may be identified, if desired, by detecting one or more biological activities characteristic of activated neutrophils. In the case of production and secretion of a cytokine such as, for example, IL-8, activated neutrophils may be identified by detecting an increase in the production and secretion of the cytokine. When the neutrophils are activated in vitro, cytokine production may be assayed, for example, by ELISA or by bioassay. When the neutrophils are activated in vivo, cytokine production may be assayed by measuring the amount of cytokine systemically (e.g., from a blood sample) or locally (e.g., from a tissue biopsy). Methods that may be used for detecting other biological activities characteristic of activated neutrophils include, for example, flow cytometry, mRNA extraction, QRT-PCR, chemotactic assays, respiratory burst assays, and phagocytosis assays. Exemplary assays are described in, for example, Hayashi et al., Blood 102(7):2660-2669 (2003).

The precise amount of IRM compound effective for activating neutrophils may vary according to factors known in the art including but not limited to the physical and chemical nature of the IRM compound; the nature of the carrier; the intended dosing regimen; whether the IRM compound is being administered in vitro or in vivo and, if in vivo, the state of the subject's immune system (e.g., suppressed, compromised, stimulated); the method of administering the IRM compound; whether a drug is being co-administered with the IRM compound and, if so, the identity, nature, and interactivity of the drug with the IRM compound; and the species to which the IRM compound is being administered. Accordingly it is not practical to set forth generally the amount that constitutes an amount of IRM compound effective for activating neutrophils for all possible applications. Those of ordinary skill in the art, however, can readily determine the appropriate amount with due consideration of such factors.

In another aspect, the present invention provides a method of treating certain conditions in a subject. As used herein, “treat” or variations thereof refer to reducing, ameliorating, or resolving, to any extent, the symptoms or signs related to a condition. “Sign” or “clinical sign” refers to an objective physical finding relating to a particular condition capable of being found by one other than the patient. “Symptom” refers to any subjective evidence of disease or of a patient's condition.

Generally, the method includes administering to the subject's neutrophils an amount of an IRM compound effective to activate the subject's neutrophils sufficiently to treat the condition. In some embodiments, the IRM compound can be administered to the subject's neutrophils in vitro. In alternative embodiments, the IRM compound can be administered to the subject's neutrophils in vivo.

When the IRM compound is administered to the subject's neutrophils in vitro, neutrophils may be collected from the subject, contacted with the IRM compound in vitro, thereby activating at least a portion of the neutrophils in the sample, and then re-introduced into the subject.

When the neutrophils are collected from the subject, the sample containing the neutrophils may include other types of cells as well. Accordingly, the sample may be enriched for neutrophils or otherwise processed before the neutrophils are activated. Alternatively, the IRM compound may be administered to an unprocessed sample. Activated neutrophils may be washed or otherwise processed before being re-introduced into the subject. In alternative embodiments, unprocessed activated neutrophils may be re-introduced into the subject. Consequently, depending upon the composition of the original sample and the degree of processing between collection and re-introduction, the cells re-introduced into the subject may include cells other than neutrophils.

An amount of IRM compound effective to activate neutrophils sufficiently to treat the condition can be any amount that either ameliorates symptoms of the condition to any degree, or slows the progression of the condition (e.g., spread of symptoms, severity of symptoms, or spread or growth of an underlying infection or tumor). In some embodiments, symptoms may be ameliorated completely so that the condition is resolved. In alternative embodiments, it may be sufficient to ameliorate one or more symptoms of the condition such as, for example, decreasing one or more of erythema, fever, pain, swelling, loss of function, bacterial load, fungal load, or tumor size.

When the IRM compound is administered to the subject's neutrophils in vivo, the IRM compound may be administered as a component of a pharmaceutical composition. Pharmaceutical compositions that include an IRM compound and methods of administering such pharmaceutical compositions are described in detail below.

The precise amount of IRM compound effective for activating neutrophils sufficiently to treat the condition may vary according to factors known in the art including but not limited to the physical and chemical nature of the IRM compound; the nature of the carrier; the intended dosing regimen; whether the IRM compound is being administered in vitro or in vivo and, if in vivo, the state of the subject's immune system (e.g., suppressed, compromised, stimulated); the method of administering the IRM compound; whether a drug is being co-administered with the IRM compound and, if so, the identity, nature, and interactivity of the drug with the IRM compound; and the species to which the IRM compound is being administered. Accordingly it is not practical to set forth generally the amount that constitutes an amount of IRM compound effective for activating neutrophils sufficiently to treat all possible conditions. Those of ordinary skill in the art, however, can readily determine the appropriate amount with due consideration of such factors.

Certain IRMs are small organic molecules (e.g., molecular weight under about 1000 Daltons, in some cases under about 500 Daltons, as opposed to large biological molecules such as proteins, peptides, and the like) such as those disclosed in, for example, U.S. Pat. Nos. 4,689,338; 4,929,624; 5,266,575; 5,268,376; 5,346,905; 5,352,784; 5,389,640; 5,446,153; 5,482,936; 5,756,747; 6,110,929; 6,194,425; 6,331,539; 6,376,669; 6,451,810; 6,525,064; 6,541,485; 6,545,016; 6,545,017; 6,573,273; 6,656,938; 6,660,735; 6,660,747; 6,664,260; 6,664,264; 6,664,265; 6,667,312; 6,670,372; 6,677,347; 6,677,348; 6,677,349; 6,683,088; 6,756,382; U.S. Patent Publication Nos. 2004/0091491; 2004/0132766; 2004/0147543; and 2004/0176367; and International Patent Application No. PCT/US04/28021 filed on Aug. 27, 2004.

Additional examples of small molecule IRMs include certain purine derivatives (such as those described in U.S. Pat. Nos. 6,376,501, and 6,028,076), certain imidazoquinoline amide derivatives (such as those described in U.S. Pat. No. 6,069,149), certain imidazopyridine derivatives (such as those described in U.S. Pat. No. 6,518,265), certain benzimidazole derivatives (such as those described in U.S. Pat. No. 6,387,938), certain derivatives of a 4-aminopyrimidine fused to a five membered nitrogen containing heterocyclic ring (such as adenine derivatives described in U.S. Pat. Nos. 6,376,501; 6,028,076 and 6,329,381; and in WO 02/08905), and certain 3-β-D-ribofuranosylthiazolo[4,5-d]pyrimidine derivatives (such as those described in U.S. Publication No. 2003/0199461).

Other IRMs include large biological molecules such as oligonucleotide sequences. Some IRM oligonucleotide sequences contain cytosine-guanine dinucleotides (CpG) and are described, for example, in U.S. Pat. Nos. 6,194,388; 6,207,646; 6,239,116; 6,339,068; and 6,406,705. Some CpG-containing oligonucleotides can include synthetic immunomodulatory structural motifs such as those described, for example, in U.S. Pat. Nos. 6,426,334 and 6,476,000. Other IRM nucleotide sequences lack CpG sequences and are described, for example, in International Patent Publication No. WO 00/75304 and Heil et al., Science (2004), vol. 303, pp. 1526-1529.

Other IRMs include biological molecules such as aminoalkyl glucosaminide phosphates (AGPs) and are described, for example, in U.S. Pat. Nos. 6,113,918; 6,303,347; 6,525,028; and 6,649,172.

Unless otherwise indicated, reference to a compound throughout this disclosure, including the appended claims, can include the compound in any pharmaceutically acceptable form, including any isomer (e.g., diastereomer or enantiomer), salt, solvate, polymorph, and the like. In particular, if a compound is optically active, reference to the compound can include each of the compound's enantiomers as well as racemic mixtures of the enantiomers.

In some embodiments of the present invention, the IRM compound can be an IRM compound that includes a 2-aminopyridine fused to a five membered nitrogen-containing heterocyclic ring. IRM compounds suitable for use in the invention include, for example, compounds having a 2-aminopyridine fused to a five membered nitrogen-containing heterocyclic ring. Such compounds include, for example, imidazoquinoline amines including but not limited to substituted imidazoquinoline amines such as, for example, amide substituted imidazoquinoline amines, sulfonamide substituted imidazoquinoline amines, urea substituted imidazoquinoline amines, aryl ether substituted imidazoquinoline amines, heterocyclic ether substituted imidazoquinoline amines, amido ether substituted imidazoquinoline amines, sulfonamido ether substituted imidazoquinoline amines, urea substituted imidazoquinoline ethers, thioether substituted imidazoquinoline amines, 6-, 7-, 8-, or 9-aryl, heteroaryl, aryloxy or arylalkyleneoxy substituted imidazoquinoline amines, and imidazoquinoline diamines; tetrahydroimidazoquinoline amines including but not limited to amide substituted tetrahydroimidazoquinoline amines, sulfonamide substituted tetrahydroimidazoquinoline amines, urea substituted tetrahydroimidazoquinoline amines, aryl ether substituted tetrahydroimidazoquinoline amines, heterocyclic ether substituted tetrahydroimidazoquinoline amines, amido ether substituted tetrahydroimidazoquinoline amines, sulfonamido ether substituted tetrahydroimidazoquinoline amines, urea substituted tetrahydroimidazoquinoline ethers, thioether substituted tetrahydroimidazoquinoline amines, and tetrahydroimidazoquinoline diamines; imidazopyridine amines including but not limited to amide substituted imidazopyridine amines, sulfonamide substituted imidazopyridine amines, urea substituted imidazopyridine amines, aryl ether substituted imidazopyridine amines, heterocyclic ether substituted imidazopyridine amines, amido ether substituted imidazopyridine amines, sulfonamido ether substituted imidazopyridine amines, urea substituted imidazopyridine ethers, and thioether substituted imidazopyridine amines; 1,2-bridged imidazoquinoline amines; 6,7-fused cycloalkylimidazopyridine amines; imidazonaphthyridine amines; tetrahydroimidazonaphthyridine amines; oxazoloquinoline amines; thiazoloquinoline amines; oxazolopyridine amines; thiazolopyridine amines; oxazolonaphthyridine amines; thiazolonaphthyridine amines; and 1H-imidazo dimers fused to pyridine amines, quinoline amines, tetrahydroquinoline amines, naphthyridine amines, or tetrahydronaphthyridine amines.

In certain embodiments, the IRM compound may be an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

As used herein, “neutrophil-activating IRM” refers to and IRM compound that is a substituted imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

As used herein, a substituted imidazoquinoline amine refers to an amide substituted imidazoquinoline amine, a sulfonamide substituted imidazoquinoline amine, a urea substituted imidazoquinoline amine, an aryl ether substituted imidazoquinoline amine, a heterocyclic ether substituted imidazoquinoline amine, an amido ether substituted imidazoquinoline amine, a sulfonamido ether substituted imidazoquinoline amine, a urea substituted imidazoquinoline ether, a thioether substituted imidazoquinoline amine, a 6-, 7-, 8-, or 9-aryl, heteroaryl, aryloxy or arylalkyleneoxy substituted imidazoquinoline amine, or an imidazoquinoline diamine. As used herein, substituted imidazoquinoline amines specifically and expressly exclude 1-(2-methylpropyl)-1H-imidazo[4,5-c]quinolin-4-amine and 4-amino-α,α-dimethyl-2-ethoxymethyl-1H-imidazo[4,5-c]quinolin-1-ethanol.

Suitable IRM compounds also may include the purine derivatives, imidazoquinoline amide derivatives, benzimidazole derivatives, adenine derivatives, and oligonucleotide sequences described above.

In some embodiments, the IRM compound may be a thiazoloquinoline amine such as, for example, 2-propylthiazolo[4,5-c]quinolin-4-amine, 2-propyl-7-(pyridin-3-yl)-thiazolo[4,5-c]quinolin-4-amine, N-[3-(4-amino-2-propylthiazolo[4,5-c]quinolin-7-yl)phenyl]methanesulfonamide, or [3-(4-amino-2-propylthiazolo[4,5-c]quinolin-7-yl)phenyl]methanol. In other embodiments, the IRM compound may be a sulfonamide substituted imidazoquinoline amine such as, for example, N-{2-[4-amino-2-(ethoxymethyl)-1H-imidazo[4,5-c]quinolin-1-yl]ethyl}methanesulfonamide. In still other embodiments, the IRM compound may be an amide substituted imidazoquinoline amine such as, for example, N-(2-{2-[4-amino-2-(2-methoxyethyl)-1H-imidazo[4,5-c]quinolin-1-yl]ethoxy}ethyl)hexadecanamide.

The IRM compound may be provided in any formulation suitable for administration to a subject. Suitable types of formulations are described, for example, in U.S. Pat. No. 5,736,553; U.S. Pat. No. 5,238,944; U.S. Pat. No. 5,939,090; U.S. Pat. No. 6,365,166; U.S. Pat. No. 6,245,776; U.S. Pat. No. 6,486,186; U.S. Patent Publication No. 2003/0199538; European Patent No. EP 0 394 026; and International Patent Publication No. WO 03/045391. The formulation may be provided in any suitable form including, but not limited to, a solution, a suspension, an emulsion, or any form of mixture. The IRM compound may be delivered in formulation with any pharmaceutically acceptable excipient, carrier, or vehicle. For example, a formulation may be delivered in a conventional topical dosage form such as, for example, a cream, an ointment, an aerosol formulation, a non-aerosol spray, a gel, a lotion, and the like. A formulation may further include one or more additives including but not limited to adjuvants, skin penetration enhancers, colorants, flavorings, fragrances, moisturizers, thickeners, and the like.

A formulation containing the IRM compound may be administered in any suitable manner such as, for example, non-parenterally or parenterally. As used herein, non-parenterally refers to administration through the digestive tract, including by oral ingestion. Parenterally refers to administration other than through the digestive tract such as, for example, intravenously, intramuscularly, transdermally, subcutaneously, transmucosally (e.g., by inhalation), or topically.

In some embodiments, the methods of the present invention include administering the IRM compound to a subject in a formulation of, for example, from about 0.0001% to about 10% (unless otherwise indicated, all percentages provided herein are weight/weight with respect to the total formulation) to the subject, although in some embodiments the IRM compound may be administered using a formulation that provides the IRM compound in a concentration outside of this range. In certain embodiments, the method includes administering to a subject a formulation that includes from about 0.01% to about 1% IRM compound, for example, a formulation that includes from about 0.1% to about 0.5% IRM compound.

In certain embodiments (e.g., embodiments in which the IRM compound is administered to a cell culture that includes neutrophils in vitro), the methods of the present invention include administering sufficient IRM compound to provide a concentration of, for example, from about 1.0 nM to about 100 mM, although in some embodiments the methods may be performed by administering the IRM compound in concentrations outside this range. In some of these embodiments, the method includes administering sufficient IRM compound to provide a concentration of from about 0.1 μM to about 1 mM. In certain embodiments, the method includes administering sufficient IRM compound to provide a concentration of from about 1 μM to about 10 μM, for example, an IRM compound concentration of from about 3 μM to about 5 μM.

In embodiments in which the IRM compound is administered to a subject, the methods of the present invention include administering sufficient IRM compound to provide a dose of, for example, from about 100 ng/kg to about 50 mg/kg to the subject, although in some embodiments the methods may be performed by administering the IRM compound in concentrations outside this range. In some of these embodiments, the method includes administering sufficient IRM compound to provide a dose of from about 10 μg/kg to about 5 mg/kg to the subject, for example, a dose of from about 100 μg/kg to about 1 mg/kg.

The dosing regimen may depend at least in part on many factors known in the art including but not limited to the physical and chemical nature of the IRM compound, the nature of the carrier, the amount of IRM compound being administered, the state of the subject's immune system (e.g., suppressed, compromised, stimulated), the method of administering the IRM compound, and the species to which the formulation is being administered. Accordingly it is not practical to set forth generally the dosing regimen effective for activating neutrophils for all possible applications. Those of ordinary skill in the art, however, can readily determine the dosing regimen with due consideration of such factors.

In some embodiments of the invention, the IRM compound may be administered, for example, from a one-time dose to multiple doses per day. In certain embodiments, the IRM compound may be administered from about once per week to about three times per day, although in some embodiments the methods of the present invention may be performed by administering the IRM compound at a frequency outside this range. In one particular embodiment, the IRM compound is administered twice per day. In an alternative embodiment, the IRM compound is administered once per day.

In some embodiments, treatment with an IRM compound can include a period of from a single, one-time dose to continuous maintenance therapy. In certain embodiments, treatment can include administering an IRM compound for from one day to about 12 weeks, although in some embodiments the methods of the present invention may be performed by administering the IRM compound for a period outside this range (e.g., continuous maintenance therapy). In one particular embodiment, the IRM compound may be administered over a period of about 10 days.

Conditions for which IRM compounds may be used as treatments include, but are not limited to:
(a) viral diseases such as, for example, diseases resulting from infection by an adenovirus, a herpesvirus (e.g., HSV-I, HSV-II, CMV, or VZV), a poxvirus (e.g., an orthopoxvirus such as variola or vaccinia, or molluscum contagiosum), a picornavirus (e.g., rhinovirus or enterovirus), an orthomyxovirus (e.g., influenzavirus), a paramyxovirus (e.g., parainfluenzavirus, mumps virus, measles virus, and respiratory syncytial virus (RSV)), a coronavirus (e.g., SARS), a papovavirus (e.g., papillomaviruses, such as those that cause genital warts, common warts, or plantar warts), a hepadnavirus (e.g., hepatitis B virus), a flavivirus (e.g., hepatitis C virus or Dengue virus), or a retrovirus (e.g., a lentivirus such as HIV);
(b) bacterial diseases such as, for example, diseases resulting from infection by bacteria of, for example, the genus Escherichia, Enterobacter, Salmonella, Staphylococcus, Shigella, Listeria, Aerobacter, Helicobacter, Klebsiella, Proteus, Pseudomonas, Streptococcus, Chlamydia, Mycoplasma, Pneumococcus, Neisseria, Clostridium, Bacillus, Corynebacterium, Mycobacterium, Campylobacter, Vibrio, Serratia, Providencia, Chromobacterium, Brucella, Yersinia, Haemophilus, or Bordetella;
(c) other infectious diseases, such chlamydia, fungal diseases including but not limited to candidiasis, aspergillosis, histoplasmosis, cryptococcal meningitis, or parasitic diseases including but not limited to malaria, pneumocystis carnii pneumonia, leishmaniasis, cryptosporidiosis, toxoplasmosis, and trypanosome infection; and
(d) neoplastic diseases, such as intraepithelial neoplasias, cervical dysplasia, actinic keratosis, basal cell carcinoma, squamous cell carcinoma, renal cell carcinoma, Kaposi's sarcoma, melanoma, leukemias including but not limited to myelogeous leukemia, chronic lymphocytic leukemia, multiple myeloma, non-Hodgkin's lymphoma, cutaneous T-cell lymphoma, B-cell lymphoma, and hairy cell leukemia, and other cancers.

Additionally, an IRM compound may be useful as a vaccine adjuvant for use in conjunction with any material that raises either humoral and/or cell mediated immune response, such as, for example, live viral, bacterial, or parasitic immunogens; inactivated viral, tumor-derived, protozoal, organism-derived, fungal, or bacterial immunogens, toxoids, toxins; self-antigens; polysaccharides; proteins; glycoproteins; peptides; cellular vaccines; DNA vaccines; autologous vaccines; recombinant proteins; glycoproteins; peptides; and the like, for use in connection with, for example, BCG, cholera, plague, typhoid, hepatitis A, hepatitis B, hepatitis C, influenza A, influenza B, parainfluenza, polio, rabies, measles, mumps, rubella, yellow fever, tetanus, diphtheria, hemophilus influenza b, tuberculosis, meningococcal and pneumococcal vaccines, adenovirus, HIV, chicken pox, cytomegalovirus, dengue, feline leukemia, fowl plague, HSV-1 and HSV-2, hog cholera, Japanese encephalitis, respiratory syncytial virus, rotavirus, papilloma virus, yellow fever, and Alzheimer's Disease.

The methods of the present invention may be performed on any suitable subject. Suitable subjects include but are not limited to animals such as but not limited to humans, non-human primates, rodents, dogs, cats, horses, pigs, sheep, goats, or cows.

### EXAMPLES

The following example has been selected merely to further illustrate features, advantages, and other details of the invention. It is to be expressly understood, however, that while the examples serve this purpose, the particular materials and amounts used as well as other conditions and details are not to be construed in a matter that would unduly limit the scope of this invention.

The compounds used in Example 1 are shown in Table 1.

TABLE 1

| Compound | Chemical Name | Reference |
|---|---|---|
| 1 | 2-propylthiazolo[4,5-c]quinolin- | U.S. 6,110,929 |
| | 4-amine | Example 12 |
| 2 | 4-amino-2-(ethoxymethyl)-α,α- | U.S. 5,352,784 |
| | dimethyl-6,7,8,9-tetrahydro-1H- | Example 91 |
| | imidazo[4,5-c]quinoline-1-ethanol | |
| 3 | N-[4-(4-amino-2-ethyl-1H-imidazo | U.S. 6,677,349 |
| | [4,5-c]quinolin-1-yl)butyl] | Example 236 |
| | methanesulfonamide | |
| 4 | 1-(2-methylpropyl)-1H-imidazo | U.S. 4,689,338 |
| | [4,5-c]quinolin-4-amine | Example 99 |
| 5 | N-(2-{2-[4-amino-2-(2-methoxyethyl)- | U.S. |
| | 1H-imidazo[4,5-c]quinolin-1-yl | 2004/0091491 |
| | ]ethoxy}ethyl)hexadecanamide | IRM3 |
| 6 | N-{2-[4-amino-2-(ethoxymethyl)- | U.S. 6,331,539^{#} |
| | 1H-imidazo[4,5-c]quinolin-1-yl] | |
| | ethyl}methanesulfonamide | |

^{#}This compound is not specifically exemplified but can be readily prepared using the synthetic methods disclosed in the cited reference.

### Example 1

Neuttrophils were enriched from human peripheral blood by HISTOPAQUE-1077 (Sigma-Aldrich Co., St. Louis, Mo.) density gradient centrifugation, and then further purified using CD15 magnetic beads (Miltenyi Biotec, Inc., Auburn, Calif.). Red blood celles in the enriched samples were lysed using an ammonium chloride lysis buffer (Biosource International Inc., Camarillo, Calif.).

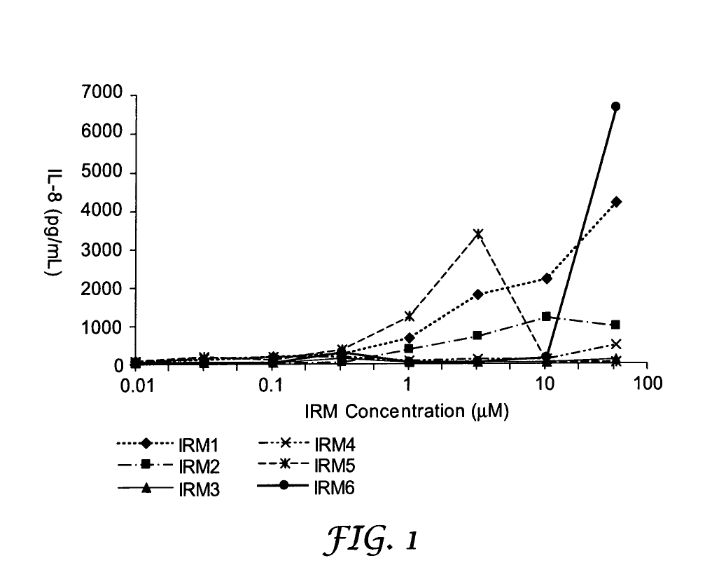

Cells were cultured overnight in heat-inactivated RPMI fetal calf serum (Biosource International Inc., Camarillo, Calif.) at 37° C., 5% CO₂. Neutrophils were stimulated by adding Compound 1 (TLR8-selective), Compound 2 (TLR7/8 agonist), Compound 3 (TLR7-selective agonist), Compound 4 (TLR7-selective agonist), Compound 5 (TLR8-selective agonist), or Compound 6 (TLR8-selective agonist) at a concentration of 0.01 μM, 0.03 μM, 0.1 μM, 0.3 μM, 1.0 μM, 3.0 μM, 10 μM, or 30 μM to the to the culture. Culture supernatants were analyzed for IL-8 production using a human-specific IL-8 BV™ immunoassay (BioVeris Corp., Gaithersburg, Md.). Results are shown in FIG. 1.

The complete disclosures of the patents, patent documents and publications cited herein are incorporated by reference in their entirety as if each were individually incorporated. In case of conflict, the present specification, including definitions, shall control.

Various modifications and alterations to this invention will become apparent to those skilled in the art without departing from the scope and spirit of this invention. Illustrative embodiments and examples are provided as examples only and are not intended to limit the scope of the present invention. The scope of the invention is limited only by the claims set forth as follows.

1. A method of activating neutrophils, the method comprising contacting neutrophils with a TLR8-selective agonist in an amount effective to activate the neutrophils.

2. The method of claim 1 wherein the neutrophils are contacted with the TLR8-selective agonist in vitro.

3. The method of claim 2 further comprising administering the activated neutrophils to a subject.

4. The method of claim 1 wherein the neutrophils are contacted with the TLR8-selective agonist in vivo.

5. The method of claim 4 wherein contacting neutrophils with the TLR8-selective agonist comprises administering a pharmaceutical composition that comprises a TLR8-selective agonist to a subject.

6. The method of claim 5 wherein the pharmaceutical composition is administered topically, intravenously, intramuscularly, transdermally, subcutaneously, or transmucosally.

7. The method of claim 5 wherein the pharmaceutical composition is administered non-parenterally.

8. The method of claim 1 wherein the TLR8-selective agonist is an IRM compound.

9. A method of treating a condition in a subject, the method comprising administering a TLR8-selective agonist to neutrophils of the subject in an amount effective to activate the neutrophils sufficiently to treat the condition.

10. The method of claim 9 wherein the TLR8-selective agonist is administered to the neutrophils in vitro in an amount effective to activate the neutrophils.

11. The method of claim 10 further comprising administering the activated neutrophils to the subject.

12. The method of claim 9 wherein the TLR8-selective agonist is administered to the neutrophils in vivo.

13. The method of claim 12 wherein administering the TLR8-selective agonist to neutrophils comprises administering a pharmaceutical composition that comprises a TLR8-selective agonist to the subject.

14. The method of claim 13 wherein the pharmaceutical composition is administered topically, intravenously, intramuscularly, transdermally, subcutaneously, or transmucosally.

15. The method of claim 13 wherein the pharmaceutical composition is administered non-parenterally.

16. The method of claim 9 wherein the TLR8-selective agonist comprises an IRM compound.

17. The method of claim 9 wherein the condition comprises infection of a subject by a pathogen.

18. The method of claim 17 wherein the pathogen is an extracellular pathogen.

19. The method of claim 18 wherein the extracellular pathogen comprises a bacterium.

20. The method of claim 19 wherein the bacterium is from the genus Escherichia, Enterobacter, Salmonella, Staphylococci, Shigella, Listeria, Aerobacter, Helicobacter, Klebsiella, Proteus, Pseudomonas, Streptococcus, Chlamydia, Mycoplasma, Pneumococcus, Neisseria, Clostridium, Bacillus, Corynebacterium, Mycobacterium, Campylobacter, Vibrio, Serratia, Providencia, Chromobacterium, Brucella, Yersinia, Haemophilus, or Bordetella.

21. The method of claim 9 wherein the condition comprises a neoplastic disease.

22. The method of claim 21 wherein the neoplastic disease comprises intraepithelial neoplasia, cervical dysplasia, actinic keratosis, basal cell carcinoma, squamous cell carcinoma, hairy cell leukemia, Karposi's sarcoma, melanoma, renal cell carcinoma, myelogeous leukemia, multiple myeloma, non-Hodgkin's lymphoma, chronic lymphocytic leukemia, cutaneous T-cell lymphoma, B-cell lymphoma, colorectal cancer, breast cancer, or lung cancer.

23. A pharmaceutical composition comprising a TLR8-selective agonist in an amount effective to activate neutrophils.

24. A method of activating neutrophils, the method comprising contacting neutrophils with a neutrophil-activating IRM compound in an amount effective to activate the neutrophils, wherein the neutrophil-activating compound comprises a substituted imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

25. The method of claim 24 wherein the neutrophils are contacted with the neutrophil-activating IRM compound in vitro.

26. The method of claim 25 further comprising administering the activated neutrophils to a subject.

27. The method of claim 24 wherein the neutrophils are contacted with the neutrophil-activating IRM compound in vivo.

28. The method of claim 27 wherein contacting neutrophils with the neutrophil-activating IRM compound comprises administering a pharmaceutical composition that comprises a neutrophil-activating IRM compound to a subject.

29. The method of claim 28 wherein the pharmaceutical composition is administered topically, intravenously, intramuscularly, transdermally, subcutaneously, or transmucosally.

30. The method of claim 28 wherein the pharmaceutical composition is administered non-parenterally.

31. The method of claim 24 wherein the neutrophil-activating IRM compound is a TLR8-selective agonist.

32. A method of treating a condition in a subject, the method comprising administering a neutrophil-activating IRM compound to neutrophils of the subject in an amount effective to activate the neutrophils sufficiently to treat the condition.

33. The method of claim 32 wherein the neutrophil-activating IRM compound is administered to the neutrophils in vitro in an amount effective to activate the neutrophils.

34. The method of claim 33 further comprising administering the activated neutrophils to the subject.

35. The method of claim 32 wherein the neutrophil-activating IRM compound is administered to the neutrophils in vivo.

36. The method of claim 35 wherein administering the neutrophil-activating IRM compound to neutrophils comprises administering a pharmaceutical composition that comprises a neutrophil-activating IRM compound to the subject.

37. The method of claim 36 wherein the pharmaceutical composition is administered topically, intravenously, intramuscularly, transdermally, subcutaneously, or transmucosally.

38. The method of claim 36 wherein the pharmaceutical composition is administered non-parenterally.

39. The method of claim 32 wherein the neutrophil-activating IRM compound comprises a TLR8-selective agonist.

40. The method of claim 32 wherein the condition comprises infection of a subject by a pathogen.

41. The method of claim 40 wherein the pathogen is an extracellular pathogen.

42. The method of claim 41 wherein the extracellular pathogen comprises a bacterium.

43. The method of claim 42 wherein the bacterium is from the genus Escherichia, Enterobacter, Salmonella, Staphylococci, Shigella, Listeria, Aerobacter, Helicobacter, Klebsiella, Proteus, Pseudomonas, Streptococcus, Chlamydia, Mycoplasma, Pneumococcus, Neisseria, Clostridium, Bacillus, Corynebacterium, Mycobacterium, Campylobacter, Vibrio, Serratia, Providencia, Chromobacterium, Brucella, Yersinia, Haemophilus, or Bordetella.

44. The method of claim 32 wherein the condition comprises a neoplastic disease.

45. The method of claim 44 wherein the neoplastic disease comprises intraepithelial neoplasia, cervical dysplasia, actinic keratosis, basal cell carcinoma, squamous cell carcinoma, hairy cell leukemia, Karposi's sarcoma, melanoma, renal cell carcinoma, myelogeous leukemia, multiple myeloma, non-Hodgkin's lymphoma, chronic lymphocytic leukemia, cutaneous T-cell lymphoma, B-cell lymphoma, colorectal cancer, breast cancer, or lung cancer.

46. A pharmaceutical composition comprising a neutrophil-activating IRM compound in an amount effective to activate neutrophils.
